(19) United States
(12) Patent Application Publication
(10) Pub. No.: US 2023/0310615 A1
(43) Pub. Date: Oct. 5, 2023

(54) OPHTHALMIC COMPOSITIONS

(71) Applicant: Alcon Inc., Fribourg (CH)

(72) Inventors: Kasey Jon Minick, Burleson, TX (US); Huagang Chen, Arlington, TX (US)

(21) Appl. No.: 18/191,957

(22) Filed: Mar. 29, 2023

Related U.S. Application Data
(60) Provisional application No. 63/362,259, filed on Mar. 31, 2022.

(51) Int. Cl. A61K 47/36; A61K 9/00; A61K 9/107; A61K 47/18; A61K 47/10; A61K 45/06

(52) U.S. Cl. CPC A61K 47/36; A61K 9/0048; A61K 9/107; A61K 47/18; A61K 47/10; A61K 45/06

(57) ABSTRACT

The invention is generally related to artificial tear composition for ophthalmic drug delivery, and more specifically to galactomannan, a cis-diol, a buffering agent and the composition is substantially free of a borate. The ophthalmic formulation having an osmolality in the range between 240 mOsm/kg and to 110 mOsm/kg.

### TECHNICAL FIELD OF THE INVENTION

The present invention relates to artificial tear composition for ophthalmic drug delivery, and more specifically to galactomannan, a cis-diol, a buffering agent and the composition is substantially free of a borate. The ophthalmic formulation having an osmolality in the range between 240 mOsm/kg and 110 mOsm/kg.

### BACKGROUND OF THE INVENTION

Dry eye syndrome (DES), also known as keratoconjunctivitis sicca (KCS), is the condition of having Dry eye. Other associated symptoms include irritation, redness, discharge, and easily fatigued eyes. Blurred vision may also occur. The symptoms can range from mild and occasional to severe and continuous. Scarring of the cornea may occur in some cases without treatment.

Dry eye symptoms may be exacerbated by consequences of environmental factors such as forced air dry heat, wind, air pollution, or reduced blinking because of watching TV, driving, laptop work, and smart phone usage. Medications, contact lens wear, surgery, smoking, and autoimmune diseases may contribute to risk factors and disease progression associated with DED, which often adversely impacts quality of life, as well as occupational productivity (Pflugfelder 2008, DEWS 2007b) leading to a deterioration in work performance (Yamada 2012).

Many ophthalmic formulations comprise compounds that provide lubricity and other desirable properties. When these formulations are instilled in the eye, the properties of such compounds can prevent undesirable problems such as bioadhesion and the formation of friction-induced tissue damage, as well as encourage the natural healing and restoration of previously damaged tissues.

Many ophthalmic formulations comprise compounds that provide lubricity and other desirable properties. When these formulations are instilled in the eye, the properties of such compounds can prevent undesirable problems such as bioadhesion and the formation of friction-induced tissue damage, as well as encourage the natural healing and restoration of previously damaged tissues.

Ophthalmic formulations have been previously described that utilize galactomannan-borate gelling systems. U.S. Pat. No. 6,403,609 to Asgharian, entitled “Ophthalmic compositions containing galactomannan polymers and borate,” describes such systems and is herein incorporated by reference in its entirety. The cross-linking of galactomannan and borate is responsible for the gel-forming behavior of the described formulations.

Traditionally, Alcon utilizes borate polyol patent (U.S. Pat. No. 6,403,609) in many of the OTC dry eye formulation and products. The borate polyol patent was held by Masood and et al. The technology worked well for many Alcon products including Systane, Systane Ultra, and Systane HA lubricant eye drops.

However, the borate polyol complexes formation requires borate or boric acid. According to recent EU Parliament Medical Device Regulations [Annex I, Section 10.4.1 of the EU Medical Device Regulation (EU 2017/745) (EU MDR)] any medical device product containing borate concentration larger than 0.10% w/w will be restricted and special CMR (carcinogenic, mutagenic, reprotoxic) warning will need to be put on the labeling by mid-2020 warning consumers that the product contains a restricted substance considered harmful to humans. The original legislation wanted to ban borate and its derivatives but that was avoided with the option to post the warning on the labeling for products that keep using these restricted substances in amounts less than 0.1% w/w.

Therefore, there are still needs for a new polyol or other type complexes, buffer systems, which can replace borate polyol complexes or using these restricted substances (borate and its derivatives) in amounts less than 0.1% w/w and still have properties to provide safety and efficacy, performance and comfort and meet medical need.

### BRIEF SUMMARY OF THE INVENTION

The present invention generally relates to an ophthalmic composition comprising a galactomannan, a buffering agent, and a cis-diol, wherein said formulation is substantially free of borate and wherein said galactomannan is present at a concentration of 0.16 w/v % to 0.19 w/v % and said buffering agent is present at a concentration of 0.1 w/v % to 0.2 w/v %, and wherein said cis-diol is sorbitol at a concentration of 0.5 to 5.0 w/v %, and wherein said galactomannan is selected from the group consisting of guar, hydroxylpropyl guar, and combinations thereof, wherein the buffering agent includes an bis-aminopolyol of formula (I) or a salt thereof

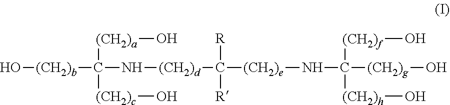

wherein a, b, c, d, e, f, g, and h are independently an integer from 1 to 6; and R and R′ are independently selected from the group consisting of —H, —CH₃, —(CH₂)₂₋₆—H, and —(CH₂)₁₋₆—OH; wherein the ophthalmic formulation having an osmolality less than 240 mOsm/kg but higher than or equal to 110 mOsm/kg.

### DETAILED DESCRIPTION

Unless defined otherwise, all technical and scientific terms used herein have the same meaning as commonly understood by one of ordinary skill in the art to which this invention belongs. Generally, the nomenclature used herein and the laboratory procedures are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well-known and commonly employed in the art.

“About” as used herein means that a number referred to as “about” comprises the recited number plus or minus 1-10% of that recited number.

“Optional” or “optionally” means that the subsequently described event or circumstance can or cannot occur, and that the description includes instances where the event or circumstance occurs and instances where it does not.

As used in this application, the term “polymer” means a material formed by polymerizing/crosslinking one or more monomers or macromers or prepolymers.

As used in this application, the term “molecular weight” of a polymeric material (including monomeric or macromeric materials) refers to the weight-average molecular weight unless otherwise specifically noted or unless testing conditions indicate otherwise.

In general, the invention is directed to an ophthalmic composition comprising a galactomannan, a buffering agent, and a cis-diol, wherein said formulation is substantially free of borate and wherein said galactomannan is present at a concentration of 0.16 w/v % to 0.19 w/v % and said buffering agent is present at a concentration of 0.1 w/v % to 0.2 w/v %, and wherein said cis-diol is sorbitol at a concentration of 0.5 to 5.0 w/v %, and wherein said galactomannan is selected from the group consisting of guar, hydroxylpropyl guar, and combinations thereof, wherein the buffering agent includes an bis-aminopolyol of formula (I) or a salt thereof

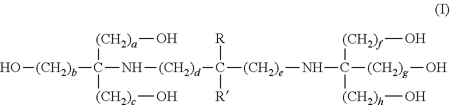

wherein a, b, c, d, e, f, g, and h are independently an integer from 1 to 6; and R and R′ are independently selected from the group consisting of —H, —CH₃, —(CH₂)₂₋₆—H, and —(CH₂)₁₋₆—OH; wherein the ophthalmic formulation having an osmolality less than 240 mOsm/kg but higher than or equal to 110 mOsm/kg.

Commercial available ophthalmic compositions, for example, Systane family products comprise guar and boric acid. Natural guar galactomannan is a water-soluble polysaccharide, which results in high viscosity even when low concentrations are dissolved in aqueous solutions. This high viscosity is in part due to its high molecular weight and the intermolecular associations occurring in the presence of borate ions. Natural guar can be treated with propylene oxide to form a more hydrophobic, surface active hydroxypropyl guar (HP-guar). Once exposed to the pH of the ocular tears and surface, approximately 7.5 pH, the HP-guar in Systane forms a “soft” gel with increased viscosity and bioadhesive properties that are designed to promote retention of the two demulcents to protect the ocular surface microenvironment.

It has been discovered the ophthalmic composition of the present application has a lower osmolality (at least 50 while has a similar viscosity comparing to an ophthalmic composition has the same composition except of replacing borate buffering with Bis-Tris-Propane. The present application can offer some advantages over a borate containing ophthalmic composition. First, the ophthalmic composition of the present application avoids the potential carcinogenic, mutagenic, reprotoxic (CMR) problems associate with the borate containing ophthalmic composition. Second, the ophthalmic composition of the present application is hypotonic and can combat any hypertonicity of tear caused by evaporation and/or disease. Third, the ophthalmic composition of the present application not only provides the most of benefits of the original Systane eye drops family products but also addresses the hypertonicity of tear caused by evaporation and/or disease. The dry eye disease is accompanied by increased tear film osmolarity and inflammation of the ocular surface. While osmolarity has become increasingly important in clinical practice as more research highlights its utility for diagnosing and monitoring DED. However, it appears commercial available dry eye drops mainly focus on increase viscosity for maintaining the formulation on the eye for a longer time and do not address the concern of hypertonicity of the dry eye symptom.

The present invention is to improve the commercial available ophthalmic compositions have an osmolality less than 240 mOsm/kg but higher than or equal to 110 mOsm/kg by replacing all or a great majority of borate in the original Systane eye drops family products with composition of galactomannan, a buffering agent, and a cis-diol and bis-aminopolyol of formula (I) or a salt thereof.

In accordance with the invention, any galactomannan polymers can be used in the present invention. As used herein, the term “galactomannan” refers to polysaccharides derived from the above natural gums or similar natural or synthetic gums containing mannose or galactose moieties, or both groups, as the main structural components. Preferred galactomannan polymers are made up of linear chains of (1-4)-β-D-mannopyranosyl units with α-D-galactopyranosyl units attached by (1-6) linkages. With the preferred galactomannan polymers, the ratio of D-galactose to D-mannose varies, but generally will be from about 1:2 to 1:4.Galactomannan polymers having a D-galactose:D-mannose ratio of about 1:2 are most preferred. Additionally, other chemically modified variations of the polysaccharides are also included in the “galactomannan polymer” definition, so long as they still have 1,3-diol moieties. For example, hydroxyethyl, hydroxypropyl and carboxymethylhydroxypropyl substitutions may be made to the galactomannan polymers. Non-ionic variations to the galactomannan polymers, such as those containing alkoxy and alkyl (C₁-C₆) groups are particularly preferred when a soft gel is desired (e.g., hydroxylpropyl substitutions). Substitutions in the non-cis hydroxyl positions are most preferred. An example of non-ionic substitution of a galactomannan polymer of the present invention is hydroxypropyl guar, with a molar substitution of about 0.4. Anionic substitutions may also be made to the galactomannan polymers. Anionic substitution is particularly preferred when strongly responsive gels are desired.

Galactomannan polymers may be obtained from numerous sources. Such sources include guar gum, locust bean gum and tara gum, as further described below. Additionally, the galactomannans may also be obtained by classical synthetic routes or may be obtained by chemical modification of naturally occurring galactomannans.

Guar gum is the ground endosperm of Cyamopisis tetragonolobus (L.) Taub. The water soluble fraction (85%) is called “guaran” (molecular weight of 220,000), which consists of linear chains of (1-4)-β-D mannopyranosyl units with α-D-galactopyranosyl units attached by (1-6) linkages. The ratio of D-galactose to D-mannose in guaran is about 1:2. The gum has been cultivated in Asia for centuries and is primarily used in food and personal care products for its thickening property. It has five to eight times the thickening power of starch. Its derivatives, such as those containing hydroxypropyl or hydroxypropyltrimonium chloride substitutions, have been commercially available for over a decade. Guar gum may be obtained, for example, from Rhone-Polulenc (Cranbury, N.J.), Hercules, Inc. (Wilmington, Del.) and TIC Gum, Inc. (Belcamp, Md.).

Locust bean gum or carob bean gum is the refined endosperm of the seed of the carob tree, Ceratonia siliqua. The ratio of galactose to mannose for this type of gum is about 1:4. Cultivation of the carob tree is old and well known in the art. This type of gum is commercially available and may be obtained from TIC Gum, Inc. (Bekamp, Md.) and Rhone-Polulenc (Cranbury, N.J.).

Tara gum is derived from the refined seed gum of the tara tree. The ratio of galactose to mannose is about 1:3. Tara gum is not produced in the United States commercially, but the gum may be obtained from various sources outside the United States.

In order to limit the extent of cross-linking to provide a softer gel characteristic, chemically modified galactomannans such as hydroxypropyl guar may be utilized. Modified galactomannans of various degree of substitution are commercially available from Rhone-Poulenc (Cranbury, N.J.). Hydroxypropyl guar with low molar substitution (e.g., less than 0.6) is particularly preferred.

In accordance with the invention, galactomannan polymer is typically present in an ophthalmic composition of the present invention at a concentration of from about 0.05 to about 5 w/v %, preferably from about 0.5 to about 2.0 w/v %, more preferably from about 0.2 to about 1.5 w/v %, and most preferably from about 0.25 to about 1.0 w/v %. Preferred galactomannan polymers of the present invention are guar and hydroxypropyl guar.

According to the present invention, the ophthalmic composition is substantially free of a borate. Substantially free of a borate refers to be less than 0.1% W/V borate, preferably less than 0.05% W/V borate, more preferably less than 0.02% W/V borate. The borate compounds which may be used in the compositions of the present invention are boric acid and other pharmaceutically acceptable salts such as sodium borate (borax), potassium borate, calcium borate, magnesium borate, manganese borate, and other such borate salts. As used herein, the term “borate” refers to all pharmaceutically suitable forms of borates. Borates are common excipients in ophthalmic formulations due to good buffering capacity at physiological pH and well known safety and compatibility with a wide range of drugs and preservatives. Borates also have inherent bacteriostatic and fungistatic properties, and therefore aid in the preservation of the compositions.

The composition of the present invention preferably contains a buffering agent. The buffering agents maintain the pH preferably in the desired range, for example, in a physiologically acceptable range of from about 6.3 to about 7.8, preferably between 6.5 to 7.6, even more preferably between 6.8 to 7.4. Any known, physiologically compatible buffering agents can be used. Suitable buffering agents as a constituent of the contact lens care composition according to the invention are known to the person skilled in the art. Examples are boric acid, borates, e.g. sodium borate, citric acid, citrates, e.g. potassium citrate, bicarbonates, e.g. sodium bicarbonate, TRIS (trometamol, 2-amino-2-hydroxymethyl-1,3-propanediol), bis-aminopolyols, phosphate buffers, e.g. Na₂HPO₄, NaH₂PO₄, and KH₂PO₄ or mixtures thereof. The amount of each buffer agent is that amount necessary to be effective in achieving a desired pH of the composition. Typically, it is present in an amount of from 0.001% to 2%, preferably from 0.01% to 1%; more preferably from about 0.05% to about 0.30% by weight, most preferably from about 0.1% to about 0.2% by weight

The preferred buffering agents are bis-aminopolyols of formula (I)

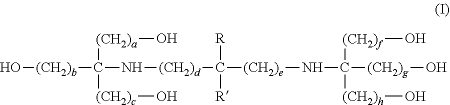

wherein a, b, c, d, e, f, g, and h are independently an integer from 1 to 6; and R and R′ are independently selected from the group consisting of —H, —CH₃, —(CH₂)₂₋₆—H, and —(CH₂)₁₋₆—OH. In the present invention, the buffering agents described by formula (I) may be provided in the form of various water-soluble salts. A most preferred bis-aminopolyol is 1,3-bis(tris[hydroxymethyl]methylamino)propane (bis-TRIS-propane).

The bis-TRIS-propane can exhibit a synergy with certain microbicides (e.g., PHMB) and fungicides, resulting in a microcidal activity significantly higher than the activity of these same active ingredients used in conjunction with other buffers. BIS-TRIS propane is described under biological buffers in Biochemicals and Reagents, Sigma-Aldrich Co., 2000-2001 edition. The specific structure of bis-TRIS-propane is shown in formula II.

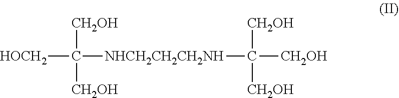

The dissociation constants for this dibasic compound are pKa₁=6.8 and pKa₂=9.5 which renders aqueous solutions of this compound useful as a buffering agent in a broad pH range from about 6.3 to 9.3. bis-TRIS-propane at a concentrations used in this invention is harmless to the eye and to known contact lens materials and is, therefore, ophthalmically compatible. Bis-Tris Propane is a buffer with an unusually wide buffering range, from approximately pH 6 to 9.5, due to its two pKa values being close in value. A solution is usually titrated to the pH desired using hydrochloric acid.

It is understood that the particular amounts of the galactomannan polymer and the bis-aminopolyols will vary. In general, the concentration of the bis-aminopolyols or the galactomannan polymer may be manipulated in order to arrive at the appropriate viscosity of the ophthalmic composition. If a high viscosity is desired, then the concentration of the bis-aminopolyols or the galactomannan polymer may be increased. Other factors may influence the gelling features of the compositions of the present invention, such as the nature and concentration of additional ingredients in the compositions, such as salts, preservatives, chelating agents, crosslinker such as Calcium chloride and magnesium chloride and so on. Generally, compositions will have a viscosity of from about 5 to 1000 cps.

In a preferred embodiment, the ophthalmic compositions of the invention may be formulated at from about 6.5 to about 8.5, preferably from about 7.0 to about 8.0. Topical formulations (particularly topical ophthalmic formulations, as noted above) are preferred which have a physiological pH matching the tissue to which the formulation will be applied or dispensed.

In another preferred embodiment, the ophthalmic composition of the present invention comprises at least one cis-diol compound at a concentration that promotes cross-linking of the galactomannan polymer to increase in viscosity and elasticity. This increase in viscosity, cross-linking, and elasticity allows for effective spreading and less blurring upon contact, yet provides long lasting lubrication and corneal surface protection. A cis-diol compound is any compound that comprise hydroxyl groups attached to adjacent carbon atoms. Exemplary cis-diol compounds include, but are not limited to, hydrophilic carbohydrates (e.g., sorbitol, mannitol), propylene glycol, glycerol, and combinations thereof. Preferred cis-diol compounds of the present invention include propylene glycol, sorbitol, mannitol and combinations thereof. The cis-diol compounds are present at concentrations of about 0.5 to 5.0 w/v %, preferably about 0.5 to 2.0 w/v % in the compositions of the present invention.

The bis-aminopolyols for example, bis-TRIS-propane in the ophthalmic compositions of the present invention is served not only as a buffering agent but also as a viscosity enhancer. The bis-TRIS-propane is not able to cross link with the galactomannan polymer as the borate does. However, the OH groups of the bis-TRIS-propane is able to form a chemical complex with the galactomannan polymer through hydrogen bond to increase viscosity of the present invention.

The ophthalmic compositions of the present invention optionally comprise a pharmaceutically acceptable divalent cation salt such as magnesium chloride. Divalent cations such as calcium generally interact with galactomannan and borate to strengthen cross-linking behavior. When present in galactomannan- and borate-containing formulations, divalent cations can increase the overall viscosity of such formulations. Divalent cations include, but are not limited to, magnesium, chloride, and zinc cations. Generally, concentrations of divalent cations should be 0 to 0.25 w/v %.

The ophthalmic compositions of the present invention may optionally comprise one or more additional excipients and/or one or more additional active ingredients. Excipients commonly used in pharmaceutical formulations include, but are not limited to, demulcents, tonicity-adjusting agents, preservatives, chelating agents, buffering agents, and surfactants. Other excipients comprise solubilizing agents, stabilizing agents, comfort-enhancing agents, polymers, emollients, pH-adjusting agents and/or lubricants. Any buffer of a variety of excipients may be used in formulations of the present invention including water, mixtures of water and water-miscible solvents, such as C1-C7-alkanols, vegetable oils or mineral oils comprising from 0.5 to 5% non-toxic water-soluble polymers, natural products, such as alginates, pectins, tragacanth, karaya gum, xanthan gum, carrageenin, agar and acacia, starch derivatives, such as starch acetate and hydroxypropyl starch, and also other synthetic products such as polyvinyl alcohol, polyvinylpyrrolidone, polyvinyl methyl ether, polyethylene oxide, preferably cross-linked polyacrylic acid and mixtures of those products.

Demulcents used with embodiments of the present invention include, but are not limited to, glycerin, polyvinyl pyrrolidone, polyethylene oxide, polyethylene glycol, polyethyleoxide-polybutyleneoxide block copolymer, polyethyleneoxide-polypropyleneoxide block copolymer, propylene glycol, polyacrylic acid, and combinations thereof. Particularly preferred demulcents are propylene glycol and polyethylene glycol 400.

Suitable tonicity-adjusting agents include, but are not limited to, mannitol, sodium chloride, glycerin, and the like. Suitable buffering agents include, but are not limited to, phosphates, acetates and the like, and amino alcohols such as 2-amino-2-methyl-1-propanol (AMP). Suitable surfactants include, but are not limited to, ionic and nonionic surfactants, though nonionic surfactants are preferred, RLM 100, POE 20 cetylstearyl ethers such as Procol® CS20 and poloxamers such as Pluronic® F68, and block copolymers such as poly(oxyethylene)-poly(oxybutylene) compounds set forth in U.S. Pat. Appl. Pub. No. 2008/0138310.

The compositions set forth herein may comprise one or more preservatives. Examples of such preservatives include p-hydroxybenzoic acid ester, sodium perborate, sodium chlorite, alcohols such as chlorobutanol, benzyl alcohol or phenyl ethanol, guanidine derivatives such as polyhexamethylene biguanide, sodium perborate, polyquaternium-1 (aka, POLYQUAD® or ONAMERM®), or sorbic acid. The use of low molecular weight amino alcohols in ophthalmic compositions is described. The compositions set forth herein may comprise low molecular weight amino alcohols (molecular weight of 60 to 200 grams/mole) to enhance the efficacy of anti-microbial preservatives. The amino alcohol is 2-amino-2-methyl-1-propanol (AMP), 2-dimethylamino-methyl-1-propanol (DMAMP), 2-amino-2-ethyl-1,3-propanediol (AEPD), 2-amino-2-methyl-1,3-propanediol (AMPD), 2-amino-1-butanol (AB), or combinations thereof. In certain embodiments, the composition may be packaged as a single dose unit so that no preservation agent is required.

Compositions of the present invention are ophthalmically suitable for application to a subject's eyes. The term “aqueous” typically denotes an aqueous formulation wherein the excipient is >50%, more preferably >75% and in particular >90% by weight of water. These drops may be delivered from a single dose ampoule which may preferably be sterile and thus render bacteriostatic components of the formulation unnecessary. Alternatively, the drops may be delivered from a multi-dose bottle which may preferably comprise a device which extracts any preservative from the formulation as it is delivered, such devices being known in the art.

The compositions of the present invention are preferably hypotonic in order to combat any hypertonicity of tears caused by evaporation and/or disease. The compositions of the present invention generally have an osmolality in the range of 110-220 mOsm/kg, and preferably have an osmolality in the range of 150-215 mOsm/kg, and more preferably have an osmolality in the range of 180-215 mOsm/kg. The ophthalmic compositions will generally be formulated as sterile aqueous solutions or sterile emulsion solutions.

In a preferred embodiment, an ophthalmic composition of the invention is an aqueous solution.

In another preferred embodiment, an ophthalmic composition of the invention is an emulsion that in addition to comprise galactomannan, a cis-diol, a buffering agent and the composition is substantially free of a borate as described above, further comprises one phospholipid and at least one ophthalmic oil dispersed throughout the continuous water or aqueous phase as small droplets that are substantially distinct and separate. It should be understood that, as used herein, the phase distinct and separate means that, at any give point in time, the droplets are distinct and separate. However, the droplets of the emulsion can combine and separate over time to maintain an average droplet size or diameter. The droplets of the emulsion of the present invention typically have an average or mean diameter no greater than about 1500 nanometers (nm), more typically no greater than about 1000 nm and still more typically no greater than about 600 nm. These droplets also typically have an average or mean diameter that is typically at least 2 nm, more typically at least 10 nm and still more typically at least 100 nm.

Particle or droplet size analyzers may be used to determine emulsion oil droplet size. For example, a Microtrac S3500 Particle Size Analyzer (Software Version 10.3.1) is a tri-laser particle size analyzer that can be used to measure emulsion oil droplet size. That particular analyzer measures laser light diffracted (scattered) from particles (e.g., droplets) in a flowing stream. The intensity and direction of the scattered light is measured by two optical detectors. Mathematical analysis of the diffraction pattern by the software generates a volume distribution of droplet size. The droplet diameter corresponding to 90% of the cumulative undersize distribution by volume is used.

It is believed that phospholipid containing in the emulsion can strengthen and/or stabilize the tear film lipid layer. By having a stabilized lipid layer, water evaporation can be reduced and symptom of dryness of the eye may be alleviated. It is also believed that phospholipids can aid in maintaining the stability of the emulsion and for reducing droplet size of the ophthalmic oil.

The emulsion of the present invention includes at least one phospholipid for aiding in maintaining the stability of the emulsion and for reducing droplet size of the oil. It is known that complex phospholipids can contain a polar group at one end of their molecular structure and a non-polar group at the opposite end of their molecular structure. A discussion of phospholipids can be found in Lehninger, Biochemistry, 2 ed., Worth Publishers, New York, pp. 279-306, incorporated herein by reference for all purposes.

Many complex phospholipids are known to the art. They differ in size, shape and the electric charge of their polar head groups. Phosphoglycerides are compounds where one primary hydroxyl group of glycerol is esterified to phosphoric acid, and the other two hydroxyl groups are esterified with fatty acids. The parent compound of the series is, therefore, the phosphoric acid ester of glycerol. This compound has an asymmetric carbon atom and, therefore, the term phosphoglycerides includes stereoisomers. All phosphoglycerides have a negative charge at the phosphate group at pH 7, and the pKₐ of this group is in the range of 1 to 2. The head groups of phosphatidylinositol, phosphatidylglycerol including diphosphatidylglycerols (having the common name cardiolipins) and the phosphatidylsugars have no electric charge, and all are polar because of their high hydroxyl group content. Because of the negative charge of the phosphate group and the absence of a charge in the head group, the net charge of each of these materials is negative, and these materials are within the scope of the invention. Suitable phospholipids are those carrying a net positive or negative charge under conditions of use. The preferred materials are those carrying a net negative charge because the negatively charged material will be repelled by the negatively charged ocular surface thereby permitting the maintenance of a relatively thick aqueous layer upon application to the eye. The most preferred phospholipid is an anionic phospholipid named dimyristoyl phosphatidylglycerol (DMPG), which is a polyol with a net negative charge. Phosphatidylglycerol or a phosphatidylinositol are other examples. Suitable phospholipid additives are disclosed in the above cited U.S. Pat. No. 4,914,088, which is fully incorporated herein by reference for all purposes.

Most phospholipids are water insoluble. However, for application to the eye, it is desirable that the phospholipid be homogeneously distributed throughout an aqueous medium. For those few phospholipids having a solubility within a useful concentration range for use as a treatment composition, a simple aqueous solution of the phospholipid in saline is satisfactory. For those phospholipids that are essentially water insoluble, an aqueous composition in the form of an emulsion may be used. An emulsion provides a treatment composition where the phase containing the phospholipid component is homogeneously distributed throughout the aqueous vehicle.

The concentration of the phospholipid in the treatment composition may vary within wide limits. A treatment composition containing the complex phospholipid in an amount as low as 0.01 weight percent provides some benefit. When the treatment composition is in the form of an emulsion, compositions containing the phospholipid in elevated concentrations approaching collapse of the emulsion into separate aqueous and phospholipid phases is possible. A clinically practical concentration range for the phospholipid in its vehicle varies from about 0.05 to 7.0 w/v % phospholipid by weight, and more preferably varies from about 0.1 and 5.0 w/v %. It should be noted that the most desired concentration for the phospholipid in the aqueous composition will vary from subject to subject.

Other additives may be present in the phospholipid treatment composition including neutral lipids such as one or more triglycerides, cholesterol esters, the natural waxes and cholesterol; higher molecular weight isoprenoids; stabilizers; preservatives; pH adjustors to provide a composition preferably having a pH between about 6 and 8 and more preferably between about 7.0 and 7.4; salt in sufficient concentration to form an isotonic composition; medicants; etc.

Examples of ophthalmic oils include without limitation any of numerous mineral oils, vegetable oil, synthetic substances, and/or animal and vegetable fats or any combination of oils. The oil can be soluble in various organic solvents such as ether but not in water. The oil phase can comprise, if desired, monoglycerides, diglycerides, triglycerides, glycolipids, glyceroglycolipids, sphingolipids, sphingo-glycolipids, fatty alcohols, hydrocarbons having a C₁₂-C₂₈ chain in length, wax esters, fatty acids, mineral oils, and silicone oils. Mineral oil is particularly preferred. A silicone oil may also be used. The oil phase can additionally include a waxy hydrocarbon, such as paraffin waxes, hydrogenated castor oil, Synchrowax HRC, Carnauba, beeswax, modified beeswaxes, microcrystalline waxes, and polyethylene waxes. The oil is typically at least 0.01 w/v %, more typically at least 0.1 w/v % and even more typically 0.8 w/v % of the emulsion. The oil is also typically no greater than about 20 w/v %, more typically no greater than about 5 w/v % and even more typically no greater than about 3 or even 1.5 w/v % of the emulsion

The emulsion will also typically include a hydrophilic surfactant (high HLB) and a hydrophobic (low HLB) surfactant. The emulsions of the present invention are most desirably used for dry eye therapeutics. However, without limitation, it is also contemplated that the emulsions may be used for drug delivery, vitamin delivery, botanical delivery, contact lens wetting and contact lens lubrication.

The emulsion of the present invention also typically incorporates two or more surfactants, which act as emulsifiers aiding in the emulsification of the emulsion. Typically, these surfactants are non-ionic. The concentration of emulsifying surfactant in the emulsion is often selected in the range of from 0.1 to 10% w/v, and in many instances from 0.5 to 5% w/v. It is preferred to select at least one emulsifier/surfactant which is hydrophilic and has an HLB value of at least 8 and often at least 10 (e.g., 10 to 18). It is further preferred to select at least one emulsifier/surfactant which is hydrophobic and has an HLB value of below 8 and particularly from 1 to 6. By employing the two surfactants/emulsifiers together in appropriate ratios, it is readily feasible to attain a weighted average HLB value that promotes the formation of an emulsion. For most emulsions according to the present invention, the average HLB value is chosen in the range of about 6 to 12, and for many from 7 to 11. For example, the HLB values for exemplary surfactants and mineral oil are as follows: hydrophobic surfactant (2.1), hydrophilic surfactant (16.9) and mineral oil (10.5).

The hydrophilic surfactant is typically present in the emulsion in an amount that is at least about 0.01 w/v %, more typically at least about 0.08 w/v % and even more typically at least about 0.14 w/v %. The hydrophilic surfactant is typically present in the emulsion in an amount that is no greater than about 1.5 w/v %, more typically no greater than about 0.8 w/v % and even more typically no greater than about 0.44 w/v %.

The hydrophilic surfactant can be a fatty acid, an ester, an ether, an acid or any combination thereof. The hydrophilic surfactant may be ionic or non-ionic, but is preferably non-ionic. Many suitable surfactants/emulsifiers are nonionic ester or ether emulsifiers comprising a polyoxyalkylene moiety, especially a polyoxyethylene moiety, often containing from about 2 to 80, and especially 5 to 60 oxyethylene units, and/or contain a polyhydroxy compound such as glycerol or sorbitol or other alditols as hydrophilic moiety. The hydrophilic moiety can contain polyoxypropylene. The emulsifiers additionally contain a hydrophobic alkyl, alkenyl or aralkyl moiety, normally containing from about 8 to 50 carbons and particularly from 10 to 30 carbons. Examples of hydrophilic surfactants/emulsifiers include ceteareth-10 to -25, ceteth-10-25, steareth-10-25, and PEG-15-25 stearate or distearate. Other suitable examples include C₁₀-C₂₀ fatty acid mono, di or tri-glycerides. Further examples include C₁₈-C₂₂ fatty alcohol ethers of polyethylene oxides (8 to 12 EO). One particularly preferred hydrophilic surfactant is polyoxyethylene-40-stearate, which is sold under the tradename MYRJ-52, which is commercially available from Nikko Chemicals.

The hydrophobic surfactant is typically present in the emulsion in an amount that is at least about 0.01 w/v %, more typically at least about 0.11 w/v % and even more typically at least about 0.16 w/v %. The hydrophobic surfactant is typically present in the emulsion in an amount that is no greater than about 10.0 w/v %, more typically no greater than about 2.0 w/v % and even more typically no greater than about 0.62 w/v %.

The hydrophobic surfactant can be a fatty acid, an ester, an ether, an acid or any combination thereof. The hydrophobic surfactant may be ionic or non-ionic, but is preferably non-ionic. The hydrophobic surfactant will typically include a hydrophobic moiety. The hydrophobic moiety can be either linear or branched and is often saturated, though it can be unsaturated, and is optionally fluorinated. The hydrophobic moiety can comprise a mixture of chain lengths, for example, those deriving from tallow, lard, palm oil sunflower seed oil or soya bean oil. Such non-ionic surfactants can also be derived from a polyhydroxy compound such as glycerol or sorbitol or other alditols. Examples of hydrophobic surfactants include, without limitation, sorbitan fatty acid esters such as sorbitan monoleate, sorbitan monostearate, sorbitan monolaurate, sorbitan monopalmitate, sorbitan monoisostearate, sorbitan trioleate, sorbitan tristearate, sorbitan sesquioleate, sorbitan sesquistearate, combinations thereof or the like. One particularly preferred hydrophobic surfactant is a sorbitan tristearate sold under the tradename SPAN-65, which is commercially available from Croda Worldwide.

The emulsion of the present invention may be formed using a variety of combining and mixing protocol and techniques known to those skilled in the art. According to one preferred embodiment, however, the ingredients are mixed and combined according to a specific protocol. In such protocol, multiple admixtures are formed and those admixtures are combined to form the emulsion. The first admixture is formed by mixing the oil and the surfactants at an elevated temperature to form an oil phase admixture. The second admixture is formed mixing the anionic phospholipid into purified water at an elevated temperature to form a water phase admixture. Thereafter, the oil phase admixture and the water phase admixture are mixed at an elevated temperature and subsequently homogenized using a homogenizer to form an initial emulsion. A third admixture is formed by mixing the galactomannan polymer with water and adjusting pH as needed to form a galactomannan polymer slurry. The galactomannan polymer slurry is then mixed with initial emulsion and form a polymer enhanced emulsion. A fourth admixture is formed by mixing any combination of the following to form a salt solution: borate, polyol, preservative and any other ingredients. The salt solution and the enhanced emulsion are then mixed followed by the addition of a sufficient quantity (Q.S.) of water and pH adjustment.

The compositions of the present invention can also be used to administer pharmaceutically active compounds and pharmaceutically acceptable salts thereof. Such compounds include, but are not limited to, anesthetic drugs, glaucoma therapeutics, pain relievers, anti-hypertensive, neuro-protective, muco-secretagogue, angiostatic, anti-angiogenesis agents, growth factors, immunosuppressant agents, anesthetic drug, anti-infectives, antiviral agents, anti-inflammatory, anti-angiogenesis agents, anti-myopia agents, anti-allergy agents, dopaminergic antagonists, proteins, and anti-microbials.

Examples of glaucoma therapeutics (or anti-glaucoma agent) include without limitation betaxolol, timolol, pilocarpine, levobetaxolol, apraclonidine, brimonidine, carbonic anhydrase inhibitors (e.g., brinzolamide and dorzolamide), and prostaglandins (e.g., travoprost, bimatoprost, and latanoprost).

Examples of anti-infective agents include without limitation ciprofloxacin and tobramycin.

Anti-inflammatory agents include non-steroidal and steroidal anti-inflammatory agents, such as triamcinolone actinide, naproxen, suprofen, diclofenac, ketorolac, nepafenac, rimexolone, tetrahydrocortisol, and dexamethasone.

Examples of antihypertensive agents include without limitation para-amino clonidine (apraclonidine).

Examples of growth factors include without limitation epidermal growth factor (EGF) and vascular endothelial growth factor (VEGF).

Examples of anti-allergy agents include without limitation olopatadine, epinastine, ketotifen, emedastine, cromolyn.

Examples of antiviral agents include without limitation ganciclovir and valganciclovir.

Examples of antimyopia agents include without limitation atropine, pirenzepine, and derivatives thereof.

Anti-angiogenesis agents include anecortave acetate (RETAANE®) and receptor tyrosine kinase inhibitors (RTKi).

Local anesthetic drugs can generally be divided into two categories based on chemical structure: “amides” and “esters.” See Ophthalmic Drug Facts '99, Facts and Comparisons, St. Louis, Mo. (1999), Ch. 3. Examples of suitable anesthetic drugs include proparacaine, lidocaine, cocaine, oxybuprocaine, benoxinate, butacaine, mepivacaine, etidocaine, dibucaine, bupivacaine, levobupivacaine, tetracaine and procaine. Most preferred are levobupivacaine, proparacaine and tetracaine.

The ophthalmic compositions of the invention can be particularly useful for delivery therapeutic agents that relieve symptoms of dry eye conditions, cooling agents, antioxidants (omega-3 and omega-6 fatty acids), nutriceuticals (e.g., vitamin A, vitamin D, vitamin E, tocopherols, vitamin K, beta-carotene), and other bioactivies for ophthalmic uses. Generally, amounts of therapeutic agent, when used, can be quite variable depending upon the agent or agents used. As such, the concentration of therapeutic agent can be at least about 0.005 w/v %, more typically at least about 0.01 w/v % and even more typically at least about 0.1 w/v %, but typically no greater than about 10 w/v %, more typically no greater than about 4.0 w/v %, still more typically no greater than about 2.0 w/v %.

Optionally, the compositions of the present invention may be formulated without a pharmaceutically active compound. Such compositions may be used to lubricate the eye or provide artificial tear solutions to treat, for example, dry eye. In general, artificial tear solutions will contain tonicity agents, polymers and preservatives, as described above. The amount of galactomannan and hydrophilic copolymer contained in the artificial tear solutions will vary, as described above, but will generally be in the amount of from 0.1 to 1.0% (w/v) and 0.1 to 4.0% (w/v), respectively.

The compositions of the invention may include additional or alternative polymeric ingredients and/or viscosity agents. Examples include, without limitation, carboxymethylcellulose, hydroxypropylmethyl cellulose, hydroxyethyl cellulose, carboxyvinyl polymer, xanthan gum, hyaluronic acid, any combinations thereof or the like.

In accordance with the invention, a composition of the present invention is administered once a day. However, the compositions may also be formulated for administration at any frequency of administration, including once a week, once every 5 days, once every 3 days, once every 2 days, twice a day, three times a day, four times a day, five times a day, six times a day, eight times a day, every hour, or greater frequency. Such dosing frequency is also maintained for a varying duration of time depending on the therapeutic regimen. The duration of a particular therapeutic regimen may vary from one-time dosing to a regimen that extends for months or years. One of ordinary skill in the art would be familiar with determining a therapeutic regimen for a specific indication.

Although various embodiments of the invention have been described using specific terms, devices, and methods, such description is for illustrative purposes only. The words used are words of description rather than of limitation. It is to be understood that changes and variations may be made by those skilled in the art without departing from the spirit or scope of the present invention, which is set forth in the following claims. In addition, it should be understood that aspects of the various embodiments may be interchanged either in whole or in part or can be combined in any manner and/or used together, as illustrated below:

The previous disclosure will enable one having ordinary skill in the art to practice the invention. Various modifications, variations, and combinations can be made to the various embodiment described herein. In order to better enable the reader to understand specific embodiments and the advantages thereof, reference to the following examples is suggested. It is intended that the specification and examples be considered as exemplary.

### Examples

TABLE 1

| INGREDIENT | Purpose | Conc. Required | |
|---|---|---|---|
| HYDROXYPROPYL GUAR 8a | Polymer | 0.16-0.19% | w/v |
| (AL12355, HP-8A) | | | |
| 6N HCl | pH adjust agent | 0.06%, | w/v |
| SORBITOL | Excipient | 1.4% | w/v |
| POLYETHYLENE GLYCOL | Demulcent | 0.4% | w/v |
| (400) | | | |
| PROPYLENE GLYCOL | Demulcent | 0.3% | w/v |
| POTASSIUM CHLORIDE | Excipient | 0.12% | w/v |
| SODIUM CHLORIDE | Excipient | 0.1% | w/v |
| POLYQUATERNIUM-1 | Preservative | 0.001% w/v + 10% xs | |
| Bis- Tris Propane (BTP) | Buffering agent | 0.14% | w/v |

| | | |
|---|---|---|
| HYDROCHLORIC ACID (1N) | pH adjust agent | adjust pH to 7.9 |
| SODIUM HYDROXIDE (1N) | pH adjust agent | adjust pH to 7.9 |
| PURIFIED WATER | Vehicle | QS to 100% |

Procedures to Prepare the Solution Ophthalmic Composition Solution of the Present Application

### I. Sterilization of HP Guar Solution

1. Pre-calibrate a 1 L clean glass bottle (+stir bar) with Purified water to 1000 g net weight, and mark the purified water level of 1 L with an adhesive tape (arrow pointed to 1 L level)
2. Add 50% of total batch volume with Milli-Q purified water.
3. While stirring, slowly add HP Guar powder into the purified water. Continue to mix for not less than 15 minutes.
4. Slowly add 1N HCl to adjust pH to ca. 6.7 (range: pH 5.5-7.0). Record pH
5. Stop agitation and let slurry stand for at least 2 hours to allow complete hydration of the HP Guar.
6. Slowly add 1N Sodium Hydroxide to Adjust pH to ca. 9.3 (range: pH 9.2-9.4). Record pH.
7. Bulk sterilize the HP Guar solution at 121° C. for 20 munities.
8. Cool the HP Guar solution rapidly to between 25-30° C.

### II. Preparation of Salt Solution

1. Place approximately 30% of the batch volume in a clean beaker with a magnetic stirrer.
2. Add and dissolve the following ingredients in the order listed. Ensure each ingredient has dissolved completely before adding the next:
   Sorbitol
   Polyethylene Glycol 400
   Propylene Glycol
   Potassium Chloride
   Sodium Chloride
   6N HCl
3. Add 1N HCl equivalent to 0.3% of the batch volume.
4. Add and dissolve the Bis Tris Propane and Polyquaternium-1.
5. Check the pH (pH should be ca 7.25).
6. Filter salt solution thru a 0.22 um sterilizing filter unit, rinse the beaker and filter with Milli-Q water two times.

### III. Finalize the Solution

1. Decontaminate the laminar flow hood and surfaces using 70% ethanol.
2. While stirring the HP Guar under the laminar flow hood, slowly open the 0.22 um sterile unit and pour the salt solution into the HP Guar solution.
3. 3. Rinse the sterile unit with Milli-Q water and bring the batch to ca 95% batch volume.
4. Check and record the pH (Target pH 7.85) adjust it if needed. Record pH.
5. QS the solution to 100% batch volume using Milli-Q water. Record final pH

TABLE 2
Systane Ultra with BTP Buffer System*
--A comparison with Systane Ultra **

| | Systane Ultra | |
|---|---|---|
| Sample | FID 112903 | BTP EYE Drops |
| Tests/Lot Number | 19643-35A | 19643-35B |
| pH | 7.9 | 7.9 |
| Osmolality | 279, 280 | 213, 215 |
| (mOsm/kg) | | |
| Viscosity (CF 42, 30 | 10.8, 10.8 | 8.6, 8.7 |
| rpm, unit: cps) | | |
| Lubricity by finger | Yes, dry faster | Yes, last longer |
| | and sticky when | btw fingers |
| | dried | |

*Composition for Systane Ultra with Bis- Tris Propane (BTP) Buffer System is shown in Table 1.
** Composition for Systane Ultra is the same as shown in table 1 except of replacing 1.4% w/v Bis Tris Propane (BTP) with 0.7% w/v boric acid.

The test results indicate that Systane Ultra with BTP Buffer System has a lower

Osmolality than Systane Ultra by 66 mOsm/Kg. This is an unexpected result because BTP is a buffering agent and not expect to have such a big effect on Osmolality of the Systane Ultra with Bis-Tris Propane (BTP) Buffer System. Composition for Systane Ultra with boric acid Buffer System is the same except of Systane Ultra with BTP Buffer System using Bis Tris Propane (BTP) as a buffering agent while Systane Ultra with boric acid Buffer System using boric acid as a buffering agent.

The test results also indicate that Systane Ultra with BTP Buffer System has a lower viscosity than Systane Ultra with boric acid buffer system by 2.1 cps. This lower viscosity for Systane Ultra with BTP Buffer System is expected because there is no boric acid in the system to cross-link with HP-guar. It is surprisingly to find out the viscosity only drop by 2.1 cps.

TABLE 3
One exemplary aqueous

| INGREDIENT | Purpose | Conc. Required | |
|---|---|---|---|
| HYDROXYPROPYL GUAR 8a | Polymer | 0.05-0.19% | w/v |
| (AL12355, HP-8A) | | | |
| 6N HCl | pH adjust agent | 0.05-0.2%, | w/v |
| SORBITOL | Excipient | 0.5-5% | w/v |
| POLYETHYLENE GLYCOL | Demulcent | 0.4% | w/v |
| (400) | | | |
| PROPYLENE GLYCOL | Demulcent | 0.3% | w/v |
| POTASSIUM CHLORIDE | Excipient | 0.12% | w/v |
| SODIUM CHLORIDE | Excipient | 0.1% | w/v |
| POLYQUATERNIUM-1 | Preservative | 0-0.001% | w/v |
| Bis Tris Propane (BTP) | Buffering agent | 0.1-0.2% | w/v |

| | | |
|---|---|---|
| HYDROCHLORIC ACID (1N) | pH adjust agent | adjust pH to 7.9 |
| SODIUM HYDROXIDE (1N) | pH adjust agent | adjust pH to 7.9 |
| PURIFIED WATER | Vehicle | QS to 100% |

Table 3 above is a formulation for one exemplary aqueous in accordance with the present invention. It is understood that the weight/volume percents in table I can be varied by ±10%, ±20%, +30%, ±90% of those weight/volume percents or more and that those variances can be specifically used to create ranges for the ingredients of the present invention. For example, an ingredient weight/volume percent of 10% with a variance of ±20% means that the ingredient can have a weight/volume percentage range of 8 to 12 w/v %.

TABLE 4
One exemplary emulsion

| | CONCENTRATION |
|---|---|
| COMPONENT | PERCENT, W/V |
| Polyquaternium-1 | 0-0.001 |
| HP-Guar | 0.05-0.2 |
| Mineral oil | 1.0 |
| 6N HCl | 0.05-0.2 |
| Bis Tris Propane (BTP) | 0.1-0.2 |
| Anionic Phospholipid | 0.005 |
| Polyoxyl 40 Stearate | 0.19-0.38 |
| Sorbitan Tristearate | 0.15-0.29 |
| Propylene Glycol | 0.6 |
| Sorbitol | 0.7 |
| Edetate Disodium | 0.025 |
| Sodium Hydroxide | Adjust pH to 7.0 |
| Hydrochloric Acid | Adjust pH to 7.0 |
| Purified Water | QS 100 |

Table 4 above is a formulation for one exemplary emulsion in accordance with the present invention. It is understood that the weight/volume percents in table I can be varied by ±10%, ±20%, 30%, ±90% of those weight/volume percents or more and that those variances can be specifically used to create ranges for the ingredients of the present invention. For example, an ingredient weight/volume percent of 10% with a variance of ±20% means that the ingredient can have a weight/volume percentage range of 8 to 12 w/v %.

galactomannan, a cis-diol, a buffering agent and the composition is substantially free of a borate

A claim for an ophthalmic emulsion of the present invention is provided as follows:

An ophthalmic emulsion, the emulsion comprising:
water forming an aqueous phase;
oil forming an oil phase;
a hydrophilic surfactant having an HLB value of from 10 to 18;
a hydrophobic surfactant having an HLB value of from 1 to 6;
a charged phospholipid;
a mucoadhesive galactomannan polymer;
a cis-diol; and
an bis-aminopolyol of formula (I) or a salt thereof,

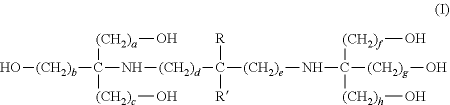

wherein a, b, c, d, e, f, g, and h are independently an integer from 1 to 6; and R and R′ are independently selected from the group consisting of —H, —CH₃, —(CH₂)₂₋₆—H, and —(CH₂)₁₋₆—OH; wherein said formulation is substantially free of borate, wherein the ophthalmic formulation having an osmolality in the range between 240 mOsm/kg and to 110 mOsm/kg.
   wherein:
      i. the oil phase is in droplets within the aqueous phase and the droplets have an average diameter that is no greater than about 1000 nm, but is at least 10 nm, wherein the average diameter is corresponding to 90% of the cumulative undersize distribution by volume measured using a Microtrac S3500 Particle Size Analyzer (Software Version 10.3.1); and
      ii. the borate and galactomannan polymer cooperatively act to form a gel upon instillation of the emulsion in an eye of an individual.

What is claimed is:

1. An ophthalmic composition comprising a galactomannan, a buffering agent, and a cis-diol, wherein said formulation is substantially free of borate and wherein said galactomannan is present at a concentration of 0.16 w/v % to 0.19 w/v % and said buffering agent is present at a concentration of 0.1 w/v % to 0.2 w/v %, and wherein said cis-diol is sorbitol at a concentration of 0.5 to 5.0 w/v %, and wherein said galactomannan is selected from the group consisting of guar, hydroxylpropyl guar, and combinations thereof, wherein the buffering agent includes an bis-aminopolyol of formula (I) or a salt thereof 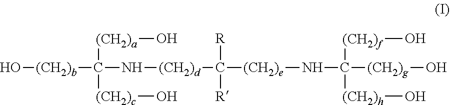
wherein a, b, c, d, e, f, g, and h are independently an integer from 1 to 6; and R and R′ are independently selected from the group consisting of —H, —CH₃, —(CH₂)₂₋₆—H, and —(CH₂)₁₋₆—OH; wherein the ophthalmic formulation having an osmolality in the range between 240 mOsm/kg and to 110 mOsm/kg.

2. The ophthalmic composition of claim 1, wherein the bis-aminopolyol is 1,3 bis(tris[hydroxymethyl]methylamino)propane (bis-TRIS-propane).

3. The ophthalmic composition of claim 1, wherein the composition has a pH value between 6.5 to 8.5.

4. The ophthalmic composition according to claim 1, wherein the ophthalmic composition comprises at least one demulcent selected from the group consisting of glycerin, polyvinyl pyrrolidone, polyethylene oxide, polyethylene glycol, polyethyleoxide-polybutyleneoxide block copolymer, polyethyleneoxide-polypropyleneoxide block copolymer, propylene glycol, polyacrylic acid, and combinations thereof.

5. The ophthalmic composition of claim 4, wherein said at least one demulcent is propylene glycol and polyethylene glycol 400.

6. The ophthalmic composition according to claim 1, wherein sorbitol at a concentration of 1.4 w/v %.

7. The ophthalmic composition according to claim 2, wherein the bis-aminopolyol is 1,3 bis(tris[hydroxymethyl]methylamino)propane (bis-TRIS-propane) at a concentration of 0.14 w/v %.

8. The ophthalmic composition according to claim 1, wherein the galactomannan is hydroxylpropyl guar.

9. The ophthalmic composition according to claim 1, wherein the ophthalmic formulation having an osmolality in the range between 220 mOsm/kg and to 170 mOsm/kg.

10. The ophthalmic composition according to claim 1, wherein the ophthalmic formulation having an osmolality in the range between 215 mOsm/kg and to 190 mOsm/kg

11. The ophthalmic composition according to claim 1, wherein the ophthalmic composition comprises an anesthetic drug, a glaucoma therapeutics, a pain reliever, an anti-hypertensive agent, a neuro-protective agent, muco-secretagogue, an angiostatic agent, an anti-angiogenesis agent, a growth factor, an immunosuppressant agent, an anesthetic drug, an anti-infective agent, an antiviral agent, an anti-inflammatory agent, an anti-angiogenesis agent, an anti-myopia agent, an anti-allergy agent, a dopaminergic antagonist, a protein, an anti-microbial, or combinations.

12. The ophthalmic composition according to claim 1, wherein the ophthalmic composition comprises a cooling agent, an antioxidant, a nutriceutical, or combinations thereof.

13. The ophthalmic composition according to claim 1, wherein the ophthalmic composition comprises omega-3 fatty acid, omega-6 fatty acid, vitamin A, vitamin D, vitamin E, tocopherols, vitamin K, beta-carotene, or combinations thereof.

14. The ophthalmic composition according to claim 1, wherein the ophthalmic composition comprises less than 0.1% W/V of borate.

15. The ophthalmic composition according to claim 1, wherein the ophthalmic composition comprises no preservative.

16. An ophthalmic emulsion, the emulsion comprising:
water forming an aqueous phase;
oil forming an oil phase;
a hydrophilic surfactant having an HLB value of from 10 to 18;
a hydrophobic surfactant having an HLB value of from 1 to 6;
a charged phospholipid;
a mucoadhesive galactomannan polymer;
a cis-diol; and
an bis-aminopolyol of formula (I) or a salt thereof, 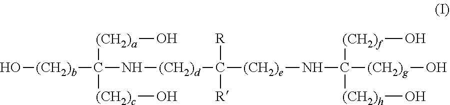
wherein a, b, c, d, e, f, g, and h are independently an integer from 1 to 6; and R and R′ are independently selected from the group consisting of —H, —CH₃, —(CH₂)₂₋₆—H, and —(CH₂)₁₋₆—OH; wherein said formulation is substantially free of borate, wherein the ophthalmic formulation having an osmolality in the range between 240 mOsm/kg and to 110 mOsm/kg.
wherein:
i. the oil phase is in droplets within the aqueous phase and the droplets have an average diameter that is no greater than about 1000 nm, but is at least 10 nm, wherein the average diameter is corresponding to 90% of the cumulative undersize distribution by volume measured using a Microtrac S3500 Particle Size Analyzer (Software Version 10.3.1); and
ii. the borate and galactomannan polymer cooperatively act to form a gel upon instillation of the emulsion in an eye of an individual.
